(19) United States
(12) Patent Application Publication
(10) Pub. No.: US 2017/0267599 A1
(43) Pub. Date: Sep. 21, 2017

(54) METHOD FOR PREPARING SOLID TRICHODERMA SEED FROM DIRECT FERMENTATION OF CROP STRAWS WITH TRICHODERMA, AND PRODUCT PREPARED BY USING THE SAME

(71) Applicant: NANJING AGRICULTURAL UNIVERSITY, Nanjing (CN)

(72) Inventors: Qirong SHEN, Nanjing (CN); Qiwei HUANG, Nanjing (CN); Dongyang LIU, Nanjing (CN); Rong LI, Nanjing (CN)

(73) Assignee: NANJING AGRICULTURAL UNIVERSITY, Nanjing (CN)

(21) Appl. No.: 15/332,859

(22) Filed: Oct. 24, 2016

(30) Foreign Application Priority Data
Mar. 16, 2016 (CN) 201610151640.1

(51) Int. Cl. C05G 3/02; A01N 63/04; C05F 9/04; C12N 1/14

(52) U.S. Cl. CPC C05G 3/02; C12N 1/14; A01N 63/04; C05F 9/04

(57) ABSTRACT

A method for preparing a solid Trichoderma seed from direct fermentation of crop straws with Trichoderma, and a product prepared by using the same. The method includes adding a liquid amino acid to the crop straws, adjusting the initial pH to 3.0-4.0, and inoculating a liquid Trichoderma seed for solid fermentation. An acidity that allows Trichoderma to grow and multiply rapidly and inhibits the growth of other non-target fungi is established in the material by adjusting its pH value, an optimum nutritional formula for Trichoderma is screened out, and an inexpensive proprietary process for preparing a solid Trichoderma seed through fermentation is established. 1% of the solid Trichoderma seed is added to a matured compost, which allows the density of Trichoderma spores in the Trichoderma amended bioorganic fertilizer to reach above 5×10⁷ spores/g, and the production increasing effect is considerable after the Trichoderma amended bioorganic fertilizer is applied.

### BACKGROUND

Technical Field

The present invention belongs to the technical field of seed fermentation, and relates to a method for preparing a solid Trichoderma seed from direct fermentation of crop straws with Trichoderma, and a product prepared by using the same.

Related Art

Trichoderma spp. is widely used in the control of soilborne wilt, which is considered as the most desirable biocontrol fungus, and has the characteristics of wide distribution, highly easy isolation and culture, and ability to inhibit the growth of various soilborne pathogens, as well as numerous biocontrol mechanisms, including hyperparasitism, antibiosis, competition, induction of resistance, and others. Particularly, Trichoderma harzianum displays a good control effect for soilborne wilt or verticillium wilt on a variety of crops including cucumber, watermelon, banana, yam, eggplant, and cotton. When colonized at the rhizosphere, the Trichoderma spp. can compete with Fusarium spp., thereby inhibiting the growth of Fusarium spp.; and rapidly degrade the toxins released by Fusarium spp., and even decompose the mycelium of Fusarium spp, thereby engulfing the Fusarium spp with the mycelium as a nutrient, thus reducing the onset of soilborne wilt on many crops. Lots of researches suggest that when Trichoderma harzianum is prepared into a bioorganic fertilizer with an organic carrier and applied to the soil, 80% or more of the soilborne wilt on crops can be controlled.

However, the reason why the Trichoderma amended bioorganic fertilizer industry cannot be developed highly is that the solid Trichoderma seed is difficult to be prepared, or the prepared solid Trichoderma seed has an inadequate spore density, and is difficult to be used in the production of a Trichoderma amended bioorganic fertilizer product having a spore density meeting the industrial standard (2×10⁷ spores/g). Because a solid Trichoderma seed with a Trichoderma spore density of 10⁹ spores/g or more needs to be produced, if the spore density in the final product is met, and then the solid seed is added to a matured compost in an amount of 3-5%. In the past, the fundamental technical route for producing a solid Trichoderma seed includes sterilizing the solid material, adjusting the humidity of the solid material, then inoculating a Trichoderma seed, and carrying out solid fermentation (multiplication) while the humidity and temperature in the solid fermentation space is maintained. The object of solid fermentation of high-density Trichoderma is generally difficult to be achieved due to the problems occurring in industrial production. For example, it is difficult to thoroughly sterilize the solid material for massive solid fermentation. Even though the solid material is sterilized thoroughly, it is difficult to control the non-target fungi in the air to enter the solid material during the solid fermentation. The rapid growth of the non-target fungi generally makes the inoculated Trichoderma difficult to grow and multiply rapidly to 10⁹ spores/g or more. Moreover, the sterilization of massive solid material in industrial production greatly increases the production cost of the manufacturer, which plus the cost for purifying the air in the fermentation space during solid fermentation, becomes a bottleneck in the economic feasibility of solid fermentation of Trichoderma. Furthermore, an optimum nutritional formula that allows the Trichoderma to grow and multiply rapidly is not found, such that the Trichoderma grows and multiplies very slowly on the solid material, and hardly becomes a dominant population to multiply rapidly and inhibit the growth of non-target fungi.

### SUMMARY

In view of the defects existing in the prior art, an object of the present invention is to provide a method for preparing a solid Trichoderma seed from direct fermentation of crop straws with Trichoderma.

Another object of the present invention is to provide a product prepared by using the same.

A further object of the present invention is to provide use of the product in the production of organic fertilizers.

The objects of the present invention may be accomplished through the following technical solutions.

A method for preparing a solid Trichoderma seed from direct fermentation of crop straws with Trichoderma comprises adding an amino acid diluent to the crop straws, then adjusting the initial pH to 3.0-4.0, aging, and inoculating a liquid Trichoderma seed for solid fermentation, to obtain a solid Trichoderma seed, wherein the temperature in the fermentation chamber is 30±2° C., and the air humidity in the chamber is 65%±5%.

The initial pH is preferably 3.0-3.5.

The amino acid diluent is obtained by diluting an amino acid hydrolyzate with water. The amino acid hydrolyzate is preferably prepared by (1) automatically smashing domestic fowl and livestock died of illness in a sealed vessel, automatically transferring all the solids and liquids to a sealed hydrolysis tank, and hydrolyzing for 2-5 hrs at an initial acid concentration c(½H₂SO₄) of 3-5 mol·L⁻¹ at 80-100° C. under 1-2 atmospheric pressure; and (2) after hydrolysis, cooling the solution in the hydrolysis tank to below 80° C., standing for layer separation, and collecting the middle-layer amino acid solution, that is, the amino acid hydrolyzate. The amino acid hydrolyzate contains about 10% (g/100 ml) or more of amino acids and various peptides.

In a preferred method of the present invention, based on the amino acid hydrolyzate, the amino acid diluent is added in an amount of 10-20 ml of the amino acid hydrolyzate per 100 g of sun dried straws, and further preferably 10 ml of the amino acid hydrolyzate per 100 g of sun dried straws.

If the pH falls outside the range of 3.0-4.0 after the amino acid diluent is added, the pH is adjusted with a base or an acid.

In a preferred embodiment of the present invention, the crop straws are selected from corn straws. After being sun dried, the corn straws have a water content of about 15% and an organic carbon content of 66%. The C/N ratio in the fermentation material is suitable when 10-20 ml of the amino acid hydrolyzate is added to 100 g of the sun dried corn straws, thus satisfying the preference of Trichoderma during growth.

In a preferred embodiment of the present invention, the corn straws have a size of 3-4 mm, and preferably 3 mm.

In the method of the present invention, the liquid Trichoderma seed is preferably inoculated to the straws in an amount of 10% (ml/100 g). The concentration of Trichoderma in the liquid Trichoderma seed is 10⁸ cfu/ml. The liquid Trichoderma seed does not have to be a spore liquid, and a fermented fresh Trichoderma liquid may be used.

In the method of the present invention, the solid fermentation time is preferably 7-10 days, and further preferably 9 days.

In the method of the present invention, the Trichoderma includes all the Trichoderma species for controlling soilborne wilt and preferably Trichoderma harzianum, and is not limited to a particular Trichoderma strain.

Things that are not detailed in the present invention may be achieved through generally accepted knowledge in the prior art or in the field.

A solid Trichoderma seed prepared according to the method of the present invention is provided.

Use of the solid Trichoderma seed according to the present invention in the production of bioorganic fertilizers is also provided.

A bioorganic fertilizer containing Trichoderma is provided, which is produced by adding 1% of the solid Trichoderma seed according to the present invention to a matured compost.

The present invention has the following innovative aspects.

1. An optimum pH value is established in the solid material at which the growth of non-target fungi is controlled, and the growth of Trichoderma is promoted.

An amino acid diluent is added to non-sterilized corn straws in an amount of 20 ml of acidolyzed amino acids at various pH values per 200 g of straws, fully mixed, and then stood for 6 hrs. Subsequently, a liquid Trichoderma seed is inoculated in an amount of 10% (ml/100 g) (the liquid Trichoderma seed does not have to be a spore liquid, and a fermented fresh Trichoderma liquid may be used), to obtain a piled-up mass having a water content of 60%. Then, solid fermentation is carried out in dark in a fermentation chamber where the temperature is 30±2° C., and the air humidity is 65%±5%. Samples are taken at various times during the fermentation, to determine the spore density of Trichoderma.

TABLE 1
Influence of different pH values on growth of Trichoderma and non-target fungi

| pH of solid | | Density of Trichoderma and non-target fungi (×10⁶ cells/g) after various fermentation time | | | | | | | | |
|---|---|---|---|---|---|---|---|---|---|---|
| material | | 1 day | 2 days | 3 days | 4 days | 5 days | 6 days | 7 days | 8 days | 9 days |
| pH | Trichoderma | 1.2 | 1.4 | 2.1 | 2.9 | 2.6 | 2.9 | 2.8 | 2.9 | 3.3 |
| 2.0 | Non-target | 2.1 | 2.4 | 1.9 | 2.0 | 2.4 | 3.0 | 2.8 | 2.4 | 2.5 |
| | fungi | | | | | | | | | |
| pH | Trichoderma | 1.3 | 2.7 | 3.9 | 4.8 | 5.2 | 6.4 | 7.6 | 8.4 | 9.9 |
| 2.5 | Non-target | 3.7 | 2.1 | 3.8 | 3.9 | 3.4 | 4.1 | 3.1 | 4.6 | 4.9 |
| | fungi | | | | | | | | | |
| pH | Trichoderma | 10.9 | 58.7 | 308 | 712 | 1368 | 3776 | 4489 | 4578 | 4601 |
| 3.0 | Non-target | 3.4 | 2.6 | 2.9 | 4.0 | 2.8 | 3.9 | 4.2 | 4.8 | 4.6 |
| | fungi | | | | | | | | | |
| pH | Trichoderma | 9.5 | 48.3 | 298 | 672 | 1485 | 4779 | 5598 | 5610 | 5809 |
| 3.5 | Non-target | 3.1 | 2.8 | 1.7 | 2.8 | 3.6 | 4.7 | 3.5 | 4.5 | 5.1 |
| | fungi | | | | | | | | | |
| pH | Trichoderma | 8.4 | 37.6 | 178 | 496 | 1254 | 4606 | 5390 | 5587 | 5630 |
| 4.0 | Non-target | 5.6 | 6.7 | 5.4 | 10.8 | 13.9 | 22.1 | 18.6 | 19.4 | 22.7 |
| | fungi | | | | | | | | | |
| pH | Trichoderma | 8.1 | 50.4 | 288 | 569 | 1042 | 1978 | 1863 | 1764 | 1856 |
| 4.5 | Non-target | 6.8 | 11.0 | 27.1 | 35.8 | 59.8 | 135 | 348 | 461 | 627 |
| | fungi | | | | | | | | | |
| pH | Trichoderma | 7.6 | 54.1 | 264 | 506 | 987 | 1007 | 1002 | 998 | 975 |
| 5.0 | Non-target | 13.8 | 59.8 | 136 | 154 | 486 | 397 | 679 | 807 | 1104 |
| | fungi | | | | | | | | | |
| pH | Trichoderma | 5.3 | 49.8 | 278 | 495 | 879 | 908 | 993 | 868 | 895 |
| 5.5 | Non-target | 32.2 | 179 | 358 | 649 | 891 | 1023 | 1579 | 1877 | 2244 |
| | fungi | | | | | | | | | |
| pH | Trichoderma | 1.9 | 39.4 | 197 | 351 | 642 | 539 | 486 | 343 | 218 |
| 6.0 | Non-target | 56.4 | 384 | 679 | 1058 | 1493 | 1946 | 2485 | 2973 | 3021 |
| | fungi | | | | | | | | | |
| pH | Trichoderma | 1.8 | 38.4 | 204 | 350 | 479 | 386 | 197 | 214 | 186 |
| 6.5 | Non-target | 108 | 667 | 1145 | 1679 | 2456 | 2978 | 3720 | 3983 | 4018 |
| | fungi | | | | | | | | | |

It can be seen from table 1 that when the pH in the initial fermentation of the solid material is 2.5 or below, the Trichoderma and non-target fungi grow and multiply very slowly, the density is slightly increased after 9 days of solid fermentation over the initial density, but does not exceed 1 order of magnitude, and particularly the density of the non-target fungi is hardly increased, suggesting that in case of a too low pH in the initial fermentation, the growth of both the Trichoderma and the non-target fungi is inhibited. When the pH in the initial fermentation of the solid material is 3.0, 3.5, and 4.0, the Trichoderma grows and multiplies at an obviously increased rate, where the growth and multiplication rate of the Trichoderma is the highest when the pH in the initial fermentation of the solid material is 3.5. After 9 days of fermentation, the density of the Trichoderma reaches 5.8×10⁹ cells/g, and the growth and multiplication rate of the non-target fungi is still very slow at pH 3.0, 3.5 and 4.0. When the pH in the initial fermentation of the solid material is above 4.5, the growth and multiplication rate of the non-target fungi is obviously increased, and the growth and multiplication rate of the Trichoderma is considerably decreased. In the test range of pH 4.5 to 6.5, the growth and multiplication rate of the non-target fungi is accelerated quickly with increasing pH. For example, when the pH in the initial fermentation of the solid material is 6.5, the density of the non-target fungi reaches 4.0×10⁹ cells/g after 9 days of fermentation. However, the density of the Trichoderma is only 1.9×10⁸ cells/g. Apparently, the decrease in the density of Trichoderma is caused by the rapid growth of the non-target fungi.

The results above suggest that once the pH in the initial fermentation of the solid material is controlled to be 3.0-4.0, and particularly the pH in the initial fermentation of the solid material is controlled to be 3.0-3.5, the growth of the non-target fungi in the solid material can be effectively inhibited, and such a pH value does not affect the growth and multiplication of the Trichoderma, such that the density of the Trichoderma in the final solid material reaches 4.6×10⁹ cells/g or more. If the solid seed is inoculated into a matured organic fertilizer in an amount of 1%, a bioorganic fertilizer product with Trichoderma at a density of 4.6×10⁷ cells/g can be obtained, which is greater than the standard (2×10⁷ cells/g) in the bioorganic fertilizer industry.

With this innovative aspects (a pH value in the initial fermentation of the solid material of 3.0-4.0 at which the growth of the non-target fungi is controlled, and the growth of Trichoderma is promoted), the enterprise has no need to sterilize the solid material, but only needs to control the pH value in the initial fermentation of the solid material, which provides an economical and effective technical process for large-scale production of bioorganic fertilizers with Trichoderma in the industry.

2. An optimum nutritional formula suitable for fermenting solid Trichoderma seeds is invented.

Trichoderma prefers fermentation materials with a high C/N ratio. However, because the carbon availability differs greatly in crop straws, lots of data needs to be obtained if inorganic nitrogen is manually added to adjust the C/N ratio in the fermentation materials. It is difficult to obtain an optimum nutritional formula for Trichoderma growth if the inorganic nitrogen is calculated and added by simply chemically determining the total organic carbon in the crop straws. In the present invention, the Trichoderma densities with various treatments are determined by carrying out solid fermentation using corn straws and inorganic nitrogen or amino acid nitrogen at various ratios, to find an optimum nutritional formula for Trichoderma growth.

TABLE 2
Influence of different nutritional formulas on Trichoderma density

| | Solid | | | | | | | | | |
|---|---|---|---|---|---|---|---|---|---|---|
| | ammonium | Density of Trichoderma (×10⁶ cells/g) after various fermentation time | | | | | | | | |
| | sulfate | 1 days | 2 days | 3 days | 4 days | 5 days | 6 days | 7 days | 8 days | 9 days |
| Corn | 1 g | 3.5 | 19.5 | 132 | 221 | 498 | 573 | 685 | 741 | 743 |
| straws | 2 g | 6.3 | 27.5 | 167 | 288 | 356 | 508 | 793 | 812 | 791 |
| 200 g | 3 g | 7.1 | 28.4 | 158 | 321 | 589 | 673 | 790 | 878 | 946 |
| | 4 g | 9.5 | 30.7 | 166 | 267 | 498 | 663 | 791 | 853 | 879 |

| | |
|---|---|
| Amino acid | Density of Trichoderma (×10⁶ cells/g) after various fermentation time |

| | hydrolyzate | 1 day | 2 days | 3 days | 4 days | 5 days | 6 days | 7 days | 8 days | 9 days |
|---|---|---|---|---|---|---|---|---|---|---|
| Corn | 10 ml | 8.6 | 52.8 | 305 | 511 | 1245 | 3489 | 4563 | 5107 | 5247 |
| straws | 20 ml | 9.2 | 60.8 | 605 | 1507 | 2486 | 3458 | 5617 | 5874 | 5903 |
| 200 g | 30 ml | 10.0 | 75.6 | 812 | 2435 | 2987 | 3978 | 4765 | 5849 | 5864 |
| | 40 ml | 11.4 | 70.2 | 715 | 2004 | 2578 | 3542 | 4568 | 5798 | 5746 |

Note:
the pH value of the solid material is 3.0-3.5 at the beginning of the fermentation, the temperature in the fermentation chamber is 30 ± 2° C., the air humidity in the chamber is 65% ± 5%, the nitrogen content of ammonium sulfate is 21%, the nitrogen content in the amino acid hydrolyzate is 1.2%, and the organic carbon content in the corn straws is 66%.

It can be seen from Table 2 that the mixture of corn straws with ammonium sulfate allows Trichoderma to grow and multiply to some degree (see ammonium sulfate blended+Trichoderma in FIG. 1). For example, after 200 g of crop straws are mixed with 2 g of ammonium sulfate and fermented for 9 days, the density of Trichoderma can reach 7-9×10⁸ cells/g. However, if such a solid seed is added to a matured organic fertilizer in an amount of 1%, the Trichoderma density in the bioorganic fertilizer product with Trichoderma is 7-9×10⁶ cells/g, which cannot meet the standard (2×10⁷ spores/g) in the bioorganic fertilizer industry. It can be seen from Table 2 that the mixture of corn straws with an amino acid hydrolyzate can obviously accelerate the growth and multiplication of Trichoderma. Particularly, when 200 g of crop straws are mixed with 40 ml of amino acid hydrolyzate and fermented for 9 days, the Trichoderma density can reach 5.9×10⁹ cells/g, and the Trichoderma completely becomes green Trichoderma spores in the later stage of growth (see amino acid blended+Trichoderma in FIG. 1). If such a solid seed is added to a matured organic fertilizer in an amount of 1%, the Trichoderma density in the bioorganic fertilizer product with Trichoderma is 5.9×10⁷ cells/g. After storage for half a year, the Trichoderma density in the product is still obviously higher than the product standard (See row 3 in Table 3). In the regulation of bioorganic fertilizer products in China, it is required that the density of the functional fungus in the product is still greater than 2×10⁷ cells/g after storage for half a year. Therefore, in the production of a solid Trichoderma seed, the Trichoderma density should be greater than 5.9×10⁹ cells/g; or otherwise, the amount of the solid Trichoderma seed added in the production of bioorganic fertilizer products with Trichoderma must be increased correspondingly, which increases the production cost of the bioorganic fertilizer manufacturers.

In this innovative aspect of the present invention, a nutritional formula suitable for high-density solid fermentation with Trichoderma is found, which is prepared by directly mixing the sun dried straws (having a water content of 15%) and the amino acid diluents at a ratio of 100 (g) of sun dried straws: 10 (ml) of the amino acid hydrolyzate and then inoculated. In case that it is ensured 10 ml of the amino acid hydrolyzate is added per 100 g of sun dried straws, generally a piled-up mass with a water content of 60-70% after inoculation of 10% Trichoderma can be obtained by adding 200-210 ml of the amino acid diluent per 100 g of sun dried straws.

TABLE 3
Change in Trichoderma density during the storage
of bioorganic fertilizers with Trichoderma

| | | Storage time of product (days) | | | | | | | | |
|---|---|---|---|---|---|---|---|---|---|---|
| | | 0 | 30 | 60 | 90 | 120 | 150 | 180 | 210 | 214 |
| Tricho- | Prod- | 3.3 | 2.9 | 2.3 | 2.0 | 1.8 | 1.7 | 1.5 | 1.4 | 1.3 |
| derma | uct I | | | | | | | | | |
| density | Prod- | 5.9 | 5.0 | 4.8 | 4.7 | 4.4 | 4.3 | 4.0 | 3.8 | 3.7 |
| (×10⁷ | uct II | | | | | | | | | |
| cells/g) | | | | | | | | | | |
| in the | | | | | | | | | | |
| product | | | | | | | | | | |

Note:
Product I refers to a Trichoderma amended bioorganic fertilizer, which is produced by inoculating a liquid Trichoderma seed to an organic fertilizer of matured pig manure in an amount of 5% and then undergoing secondary fermentation; and Product II is produced by adding the solid Trichoderma seed of the present invention to an organic fertilizer of matured pig manure in an amount of 1% and then undergoing secondary fermentation.

The industrial standard for the density of functional Trichoderma in bioorganic fertilizer products is 2×10⁷ cells/g.

3. The size of the straws for solid fermentation with Trichoderma is established.

Oxygen is needed in solid fermentation with Trichoderma. However, oxygen cannot be supplemented by stirring during solid fermentation, because stirring may break the Trichoderma mycelium to produce spores, thus inhibiting the growth of Trichoderma mycelium. To ensure the solid fermentation matrix to have a certain concentration of oxygen, the solid material is generally processed to be slightly coarse in production. However, if the solid material is too coarse, the growth and multiplication of Trichoderma is influenced because it is difficult to be made use, and an aesthetically pleasant high-quality bioorganic fertilizer product is hardly to be obtained after a too coarse solid seed is blended into the bioorganic fertilizer. In the present invention, a size of the crop straws is established, which allows the Trichoderma to efficiently make use of the crop straws, and allows the size of the bioorganic fertilizer after the solid Trichoderma seed is blended to meet the product requirement.

TABLE 4
Influence of straw size on solid fermentation with Trichoderma

| Size of corn | Density of Trichoderma (×10⁶ cells/g) after various fermentation time | | | | | | | | |
|---|---|---|---|---|---|---|---|---|---|
| straws | 1 days | 2 days | 3 days | 4 days | 5 days | 6 days | 7 days | 8 days | 9 days |
| 1 mm | 5.4 | 38.1 | 129 | 198 | 247 | 316 | 457 | 498 | 501 |
| 2 mm | 6.4 | 40.1 | 203 | 317 | 486 | 508 | 645 | 722 | 706 |
| 3 mm | 12.0 | 73.6 | 798 | 2374 | 3012 | 3985 | 4803 | 5748 | 5905 |
| 4 mm | 12.4 | 69.1 | 725 | 1007 | 2147 | 2879 | 3069 | 3601 | 3841 |
| 5 mm | 8.4 | 41.7 | 574 | 450 | 679 | 701 | 823 | 956 | 991 |

Note:
the pH value of the solid material is 3.0-3.5 at the beginning of the fermentation, the temperature in the fermentation chamber is 30 ± 2° C., and the air humidity in the chamber is 65% ± 5%.

It can be seen from Table 4 that when the size of the corn straws is 3 mm, the fermentation with Trichoderma is the most desirable, where the density of the Trichoderma spores reaches 5.9×10⁹ spores/g at Day 9. When the straw size is less than 2 mm, the density of the Trichoderma spores is only 5-7×10⁸ spores/g at Day 9 during fermentation because a too small size affects the vent performance of the fermentation matrix. When the straw size is greater than 5 mm, the growth and multiplication of Trichoderma is affected due to the rapid water loss and the decreased utilization of straws, where the density of the Trichoderma spores is 9.9×10⁸ spores/g at Day 9 during fermentation, and thus a product thus obtained cannot be ready for use as a solid Trichoderma seed.

In this innovative aspect, it is determined that the size of the straws for fermentation of solid Trichoderma seed is 3-4 mm, and preferably 3 mm, and such as straw size can be easily achieved during industrial production.

Beneficial Effects:

In view of the bottleneck problem existing in industrialized solid fermentation with Trichoderma, instead of the conventional thought to sterilize the solid material, an acidity that allows Trichoderma to grow and multiply rapidly and inhibits the growth of other non-target fungi is established in the solid fermentation material by adjusting the pH value of the solid material in the present invention, an optimum nutritional formula for Trichoderma is screened out, and an inexpensive proprietary process for preparing a solid Trichoderma seed through fermentation is creatively established. 1% of the solid Trichoderma seed prepared according to the method of the present invention is added to a matured compost, which allows the density of Trichoderma spores in the Trichoderma amended bioorganic fertilizer to reach above 5×10⁷ spores/g, and the production increasing effect is considerable after the Trichoderma amended bioorganic fertilizer is applied. It can be seen that the method of the present invention is suitable for producing accepted bioorganic fertilizer products with Trichoderma by ordinary bioorganic fertilizer manufacturers.

### BRIEF DESCRIPTION OF THE DRAWINGS

FIG. 1 shows results from fermentation with Trichoderma after different liquids are added to the straws.

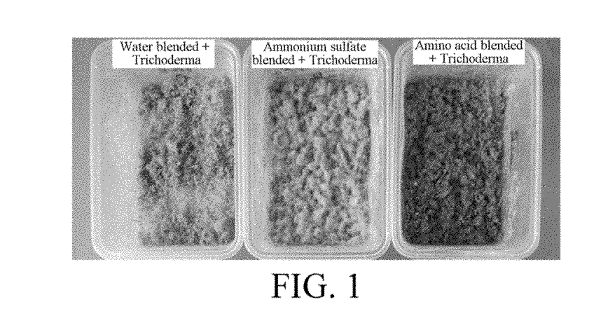

In FIG. 1, water blended+Trichoderma means that the straws are blended with water and then Trichoderma is inoculated; ammonium sulfate blended+Trichoderma means that the straws are blended with an aqueous ammonium sulfate solution and then Trichoderma is inoculated; and amino acid blended+Trichoderma means that the straws are blended with an amino acid diluent and then Trichoderma is inoculated.

Information about deposit of sample of biological material

SQR-T037, taxonomically designated as Trichoderma harzianum, is deposited on Sep. 22, 2009 in China General Microbiological Culture Collection Center (CGMCC) (Institute of Microbiology, Chinese Academy of Sciences, Datun, Chaoyang District, Beijing, China, 100101) under CGMCC Accession No. 3308.

### DETAILED DESCRIPTION

The technical solution of the present invention is described with Trichoderma harzianum SQR-T037 as an example; however, the protection scope of the present invention is not limited thereto. Practically, effects comparable to that in Example 1 are obtained by the inventors through solid fermentation with a variety of self-isolated and commercially available Trichoderma strains following the method according to the present invention. In view of the requirement for sufficient disclosure of a patent, the fermentation method of the solid Trichoderma seed according to the present invention is described merely with Trichoderma harzianum SQR-T037 as an example.

### Example 1

A method for preparing a solid Trichoderma seed from direct fermentation of crop straws with Trichoderma was provided. Amino acid diluent was added to sun dried corn straws with a size of 3 mm, a water content of 15%, and an organic carbon content of 66% in an amount of 200 ml of the amino acid diluent per 100 g of the sun dried straws (that is, 10 ml of an amino acid hydrolyzate was added per 100 g of the sun dried straws), adjusted to pH 3.5, and aged for 12 hrs. Then, a 10% liquid Trichoderma SQR-T037 seed was inoculated, to obtain a piled-up mass having a water content of 60-70%. The piled-up mass is subjected to solid fermentation, where the temperature in the fermentation chamber was 30±2° C., and the air humidity in the chamber was 65%±5%. After 9-day fermentation, a solid Trichoderma seed was obtained, in which the Trichoderma density was 5.8×10⁹ cells/g, and the non-target fungi density was 5.1×10⁶ cells/g.

### Example 2

To a matured pig manure compost, 1% of the solid Trichoderma seed prepared following the method according to the present invention was added for solid fermentation, during which the piled-up mass was stirred 1-2 times every day, such that the temperature during solid fermentation was not higher than 60° C. The fermentation was completed after 6-7 days, to obtain a Trichoderma amended bioorganic fertilizer. The field bioefficacy tests on Chinese cabbage were carried out in Nanjing and Nantong, Jiangsu respectively. The field bioefficacy test results show that in the case of equivalent nutrient input, the production is increased by 23.4% and by 9.3% in a yellow-brown-soil vegetable field in Nanjing treated with the Trichoderma amended bioorganic fertilizer vs a chemical fertilizer and an amino acid organic fertilizer (see Table 5); and the production is increased by 5.6% and by 10.5% in a slightly salinizated soil in Binhai, Nantong treated with the Trichoderma amended bioorganic fertilizer vs a chemical fertilizer and an amino acid organic fertilizer (see Table 6). The field bioefficacy test results suggest that the production increasing effect of the Trichoderma amended bioorganic fertilizer is significant than that of chemical fertilizer, which sets a material base for the generalization of the Trichoderma amended bioorganic fertilizer.

TABLE 5
Effect of application of Trichoderma amended bioorganic
fertilizer on the production of Chinese cabbage (Nanjing
Institute of vegetable science, Hengxi, Nanjing, 2015)

| | | | Rate of production |
|---|---|---|---|
| | | Rate of production | increase compared |
| | | increase compared | with amino acid |
| Treat- | Average | with chemical | organic |
| ment | (kg/mu) | fertilizer (%) | fertilizer (%) |
| CF | 7417.3 ± 747.3 | — | — |
| OF | 8368.4 ± 562.0 | 12.8 | — |
| BIO | 9149.6 ± 336.6 | 23.4 | 9.3 |

CF: Applied with chemical fertilizer
OF: Applied with amino acid organic fertilizer, which is equivalent to the chemical treatment in nutrients
BIO: Applied with Trichoderma amended bioorganic fertilizer, which is equivalent to the chemical treatment in nutrients

TABLE 6
Effect of application of Trichoderma amended bioorganic fertilizer
on the production of Chinese cabbage (Haian, Nantong, 2015)

| | | | | Rate of |
|---|---|---|---|---|
| | | Rate of | Rate of | production |
| | | production | production | increase |
| | | increase | increase | compared |
| | | compared | compared | with amino |
| Treat- | Average | with fertilizer | with chemical | acid organic |
| ment | (kg/mu) | naïve (%) | fertilizer (%) | fertilizer (%) |
| CK | 8852 ± 63.3 | — | — | — |
| CF | 9442 ± 182.8 | 6.7 | — | — |
| OF | 9029 ± 158.3 | 2.0 | −4.4 | — |
| BIO | 9973 ± 182.8 | 12.7 | 5.6 | 10.5 |

CK: blank without fertilizer applied
CF: Applied with chemical fertilizer
OF: Applied with amino acid organic fertilizer, which is equivalent to the chemical treatment in nutrients
BIO: Applied with Trichoderma amended bioorganic fertilizer, which is equivalent to the chemical treatment in nutrients

1. A method for preparing a solid Trichoderma seed from direct fermentation of crop straws with Trichoderma, comprising adding an amino acid diluent to the crop straws, then adjusting the initial pH to 3.0-4.0, aging, and inoculating a liquid Trichoderma seed for solid fermentation, to obtain a solid Trichoderma seed, wherein the temperature in the fermentation chamber is 30±2° C., and the air humidity in the chamber is 65%±5%.

2. The method according to claim 1, wherein the initial pH is 3.0-3.5.

3. The method according to claim 1, wherein the amino acid diluent is obtained by diluting an amino acid hydrolyzate with water; and the amino acid hydrolyzate is prepared by (1) automatically smashing domestic fowl and livestock died of illness in a sealed vessel, automatically transferring all the solids and liquids to a sealed hydrolysis tank, and hydrolyzing for 2-5 hrs at an initial acid concentration c(½H₂SO₄) of 3-5 mol·L⁻¹ at 80-100° C. under 1-2 atmospheric pressure; and (2) after hydrolysis, cooling the solution in the hydrolysis tank to below 80° C., standing for layer separation, and collecting the middle-layer amino acid solution, that is, the amino acid hydrolyzate.

4. The method according to claim 3, wherein based on the amino acid hydrolyzate, the amino acid diluent is added in an amount of 10-20 ml of the amino acid hydrolyzate per 100 g of sun dried straws.

5. The method according to claim 4, wherein after being mixed, the crop straws and the amino acid diluent are aged for 6-24 hrs and then 10% (ml/100 g) of the liquid Trichoderma seed is inoculated, to obtain a piled-up mass having a water content of 60-70%.

6. The method according to claim 1, 4 or 5, wherein the crop straws are corn straws.

7. The method according to claim 5, wherein the concentration of Trichoderma in the liquid Trichoderma seed is 10⁸ cfu/ml.

8. A solid Trichoderma seed prepared according to the method as set forth claim 1.

9. A method of using the solid Trichoderma seed according to claim 8 in the production of bioorganic fertilizers.

10. A bioorganic fertilizer containing Trichoderma, obtainable by adding 1% of the solid Trichoderma seed according to claim 8 to a matured compost.
